SEQUENCE LISTING (4 sequences)

SEQ ID NO: 1 - DNA, 660 bp, Artificial Sequence
  Synthetic
atgtcgaattcttatgactcctccagtatcaaagtcctgaaagggctggatgcggtgcgtaagcgcccgggtatgtatatcggcgacacggatgacggcaccggtctgcaccacatggtattcgaggtggtagataacgctatcgacgaagcgctcgcgggtcactgtaaagaaattatcgtcaccattcacgccgataactctgtctctgtacaggatgacgggcgcggcattccgaccggtattcacccggaagagggcgtatcggcggcggaagtgatcatgaccgttctgcacgcaggcggtaaatttgacgataactcctataaagtgtccggcggtctgcacggcgttggtgtttcggtagtaaacgccctgtcgcaaaaactggagctggttatccagcgcgagggtaaaattcaccgtcagatctacgaacacggtgtaccgcaggccccgctggcggttaccggcgagactgaaaaaaccggcaccatggtgcgtttctggcccagcctcgaaaccttcaccaatgtgaccgagttcgaatatgaaattctggcgaaacgtctgcgtgagttgtcgttcctcaactccggcgtttccattcgtctgcgcgacaagcgcgacggcaaagaagaccacttccactatgaagcc

SEQ ID NO: 2 - DNA, 888 bp, Artificial Sequence
  Synthetic
cctactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttag

SEQ ID NO: 3 - DNA, 1557 bp, Artificial Sequence
  Synthetic
atgtcgaattcttatgactcctccagtatcaaagtcctgaaagggctggatgcggtgcgtaagcgcccgggtatgtatatcggcgacacggatgacggcaccggtctgcaccacatggtattcgaggtggtagataacgctatcgacgaagcgctcgcgggtcactgtaaagaaattatcgtcaccattcacgccgataactctgtctctgtacaggatgacgggcgcggcattccgaccggtattcacccggaagagggcgtatcggcggcggaagtgatcatgaccgttctgcacgcaggcggtaaatttgacgataactcctataaagtgtccggcggtctgcacggcgttggtgtttcggtagtaaacgccctgtcgcaaaaactggagctggttatccagcgcgagggtaaaattcaccgtcagatctacgaacacggtgtaccgcaggccccgctggcggttaccggcgagactgaaaaaaccggcaccatggtgcgtttctggcccagcctcgaaaccttcaccaatgtgaccgagttcgaatatgaaattctggcgaaacgtctgcgtgagttgtcgttcctcaactccggcgtttccattcgtctgcgcgacaagcgcgacggcaaagaagaccacttccactatgaaggcggcggatcccctactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttag

SEQ ID NO: 4 - DNA, 1557 bp, Artificial Sequence; identical to SEQ ID NO: 3
  Synthetic

(19) United States
(12) Patent Application Publication
(10) Pub. No.: US 2014/0377870 A1
(43) Pub. Date: Dec. 25, 2014

(54) METHOD FOR INSERTING GENETIC MATERIAL INTO GENOMIC DNA

(71) Applicants: Bertram JACOBS, Tempe, AZ (US); Stacy White, Chandler, AZ (US); Kip Conwell, Chicago, IL (US); Jeffrey Langland, Chandler, AZ (US); James Jancovich, Tempe, AZ (US); Karen Kibler, Scottsdale, AZ (US)

(72) Inventors: Bertram JACOBS, Tempe, AZ (US); Stacy White, Chandler, AZ (US); Kip Conwell, Chicago, IL (US); Jeffrey Langland, Chandler, AZ (US); James Jancovich, Tempe, AZ (US); Karen Kibler, Scottsdale, AZ (US)

(73) Assignee: Arizona Board of Regents, a body corporate of the State of Arizona, acting for and on behalf of Ariz, Scottsdale, AZ (US)

(21) Appl. No.: 14/473,219

(22) Filed: Aug. 29, 2014

Related U.S. Application Data
(62) Division of application No. 13/927,673, filed on Jun. 26, 2013.
Application No. 13/927,673 is a continuation of application No. 13/697,306, now abandoned.
(60) Provisional application No. 61/352,752, filed on Jun. 8, 2010.

(51) Int. Cl. C12N 15/86; C12N 15/82; C12N 15/79; C12N 15/80; C12N 15/85; C12N 15/70

(52) U.S. Cl. CPC C12N 15/86; C12N 15/85; C12N 15/70; C12N 15/79; C12N 15/80; C12N 15/82; C12N 2800/40
USPC 435/456; 435/463; 435/471; 435/468

(57) ABSTRACT

The present invention provides reagents and methods for improved homologous recombination.

### RELATED APPLICATIONS

This application is a divisional of U.S. application Ser. No. 13/927,673 filed Jun. 26, 2013, which is a continuation of U.S. application Ser. No. 13/697,306, which claims the benefit of PCT application PCT/US11/39599, which claims priority to U.S. Provisional Patent Application Ser. No. 61/352,752 filed Jun. 8, 2010, all of which are incorporated by reference herein in their entirety.

### STATEMENT OF GOVERNMENT INTEREST

This work was supported under grant number AI052347 from the National Institutes of Health. The U.S. government has certain rights in the invention.

### BACKGROUND OF THE INVENTION

Homologous recombination is the process by which similar DNA sequences exchange information with one another. Foreign or altered DNA is flanked with homologous sequences to a specific locus of the receiving genomic DNA, which allows for a targeted insertion of the foreign/altered genetic material. This technology has led to the development of genetically modified organisms, including mammals, fungi and viruses, which have proven invaluable in the advancement of understanding biological systems. The generation of recombinant viruses has become commonplace in the field of virology and has provided a platform to study protein functions, pathogenic determinants and potential vaccine candidates. This technology has extended into more complex systems such as whole animal models. The use of homologous recombination and embryonic stem (ES) cells permits the expression of a modified gene in a particular locus in order to study the phenotypic consequences in a whole animal model. The principle behind the process is straightforward, however, homologous recombination based integration occurrence is quite low and therefore improved methods to isolate the genetically modified organism is critical.

There are many different methods available to isolate recombinants generated through homologous recombination. Since homologous recombination is a rare event, various selection markers are used for isolation of recombinants. Positive selection provides a means to enrich the population of clones that have taken up foreign DNA. These markers typically confer antibiotic resistance, such as neomycin, hygromycin, puromycin, and blasticidin S resistance cassettes. Though a variety of positive selection markers exists, very few exist for negative selection. Negative selection markers are necessary to select against random integrations during homologous recombination and/or elimination of marker genes. The herpes simplex-thymidine kinase (HSV-TK) gene has gained widespread use as a negative selectable marker. The gene product converts ganciclovir (GCV), into a cytotoxic nucleoside analog. In the presence of GCV, cells expressing HSV-TK will not replicate, as this method inhibits DNA synthesis by incorporating altered nucleotides that results in DNA chain termination. Currently, this is the primary negative selection marker used. However, this system uses a nucleoside analog thereby making it potentially mutagenic, resulting in random mutations elsewhere in the genomic DNA. In addition, GCV has been shown to exert nonspecific toxicity in cells not expressing the HSV-TK gene and reduce the totipotency of ES cells. Furthermore, this system shows variability in effectiveness, resulting in high background. Other selection cassettes used, but to a lesser extent, are hypoxanthine phosphoribosyltransferase (HPRT) and or adenine phosphoribosytransferase (ARPT), both of which require special cell lines (HRPT −/− and ARPT −/− cells), which are not readily available.

### BRIEF SUMMARY OF THE INVENTION

The present invention provides reagents and methods for inserting genetic material into genomic DNA. In a first aspect, the present invention provides nucleic acid constructs comprising (a) a first nucleic acid encoding GyrB-PKR; (b) a first homologous recombination site flanking the first nucleic acid; and (c) a second homologous recombination site flanking the first nucleic acid. In one embodiment, the constructs further comprise a second nucleic acid encoding a second selection marker operatively linked to the nucleic acid encoding GyrB-PKR, wherein the first homologous recombination site and the second homologous recombination site flank the second nucleic acid.

In another aspect, the present invention provides expression vectors comprising the nucleic acid construct of any embodiment or combination of embodiments of the invention, wherein the expression vector comprises nucleic acid control sequences operatively linked to the nucleic acid construct. In various embodiments, the expression vector comprises a plasmid or a viral vector.

In a further aspect, the present invention provides host cells comprising the nucleic acid constructs, and/or expression vectors of any embodiment or combination of embodiments of the invention. In one embodiment, the host cell comprises the nucleic acid construct of any embodiment or combination of embodiments of the invention, stably integrated into its genome.

In a still further aspect, the present invention provides kits comprising (a) the nucleic acid construct, the expression vector, and/or the host cell of any embodiment or combination of embodiments of the invention; and (b) a cloning vector, comprising the first homologous recombination site and the second homologous recombination site flanking a cloning site.

In another embodiment, the present invention provides methods for homologous recombination, comprising (a) expressing in a host cell a first expression vector and/or a nucleic acid construct according to any embodiment or combination of embodiments of the invention; (b) expressing in the host cell a second expression vector comprising a gene of interest flanked by the first homologous recombination site and the second homologous recombination site; and (c) culturing the cells in medium comprising coumermycin under conditions suitable to cause host cell death if homologous recombination has not occurred.

### BRIEF DESCRIPTION OF THE FIGURES

FIG. 1. Coumermycin/GyrB-PKR system as a negative selection marker for isolation of VACV recombinants. (A) Schematic Representation of the Coumermycin/PKR system. Coumermycin induced dimerization of GyrB-PKR fusion protein results in activation of the fused PKR catalytic domain. The GyrB-PKR fusion protein consists of the first 220 amino acids of the E. coli gyrase B coumermycin dependent dimerization domain (GyrB-DD) fused to residues 258-551 of the catalytic domain of human PKR(PKR-KD). The GyrB domain binds to coumermycin in a 2:1 ratio resulting in the dimerization and activation of the kinase domain of PKR. This leads to the phosphorylation of translation initiation factor, eIF2α, causing an inhibition of protein synthesis. (B) Expression of GyrB-PKR and GyrB-PKRK296H in recombinant VACV. RK13 cells were infected at an MOI of 5 with the indicated VACV mutants. At 6 hpi, cell extracts were harvested and subjected to Western blot analysis using antibodies that recognize human PKR.

FIG. 2. VACV expressing GyrB-PKR is sensitive to the effects of coumermycin. (A) Coumermycin sensitivity of VACVΔE3L::GyrB-PKR. RK13 cells were pretreated with increasing doses of coumermycin A1 for 16 hours and then infected with 50-100 pfu of the indicated viruses. At 48 hpi, cells were stained with crystal violet and the plaques quantitated.

(B) Coumermycin sensitivity of VACVΔJ2R::GyrB-PKR. RK13 cells were mock treated or treated with 100 ng/ml of coumermycin A1 for 24 hours. The cells were infected with ten-fold serial dilutions of VACVΔJ2R::GyrB-PKR. At 48 hpi, cells were stained with crystal violet and the number of plaques quantitated.

FIG. 3. Western blot analysis of eIF2α phosphorylation in RK13 cells infected with VACVΔE3L::GyrB-PKR in the presence of coumermycin. RK13 cells were pretreated with 100 ng/ml of coumermycin A1 for 16 hours and then infected at an MOI of 5 with the indicated viruses. At 6 hours post infection, cell extracts were harvested and subjected to Western blot analysis using antibodies against ser51-phospho-eIF2α.

FIG. 4. Coumermycin/GyrB-PKR selection of VACV recombinants. (A) Parental cmr^{s} VACV expressing GyrB-PKR in the locus of interest undergoes homologous recombination with foreign genetic material flanked by homologous sequences to the locus of interest. Only recombinants that replaced the negative selection marker are resistant (cmr^{r}) to the effects of coumermycin, allowing for their enrichment in the presence of the antibiotic. (B) VACVΔE3L::LacZ was generated by in vivo recombination between pMPLacZ and parental virus VACVΔE3L::GyrB-PKR. Progeny virus was then plagued out in RK13 cells in the presence or absence of coumermycin. A comparison of the number of recombinants (blue plaques) to parental virus (clear plaques) in the presence or absence of coumermycin was determined. VACVΔE3L::GyrB-PKR and recombinant VACVΔE3L::LacZ were screened using X-gal and neutral red staining.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

In a first aspect, the present invention provides nucleic acid constructs, comprising
a) a first nucleic acid encoding GyrB-PKR;
b) a first homologous recombination site flanking the first nucleic acid; and
c) a second homologous recombination site flanking the first nucleic acid.

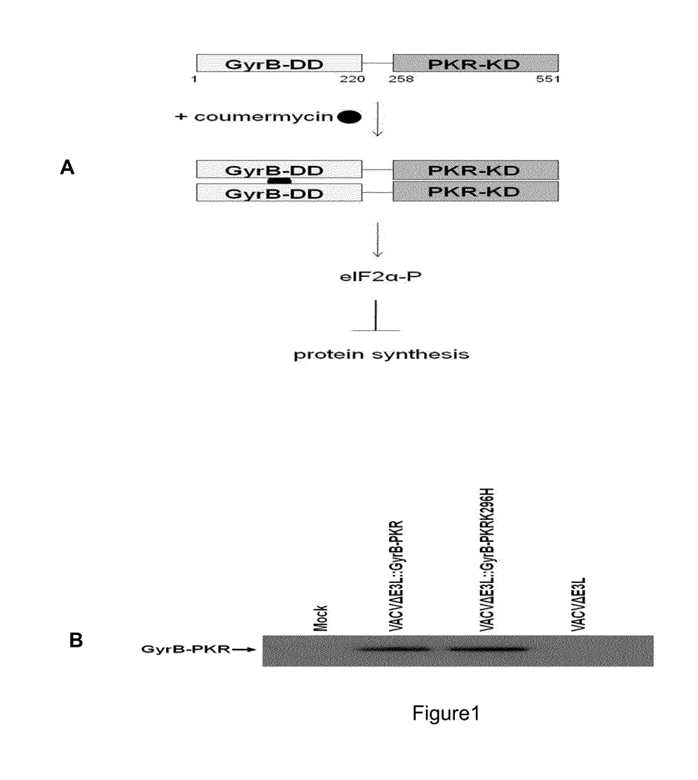

This invention provides reagents and methods that improve on existing negative selection markers used in the generation of recombinant organisms. Currently, the HSV-TK negative selection marker is the most widely used marker for negative selection. However, this selection method has limitations: The HSV-TK uses GCV, a nucleoside analog. Therefore, this selection scheme inhibits replication of DNA by the incorporation of an altered nucleotide, which is potentially mutagenic, resulting in the increase of second site mutations. GCV also reduces the totipotentcy of ES cells and has been shown to exert nonspecific toxicity in cells not expressing the HSV-TK gene. The nucleic acid constructs of this first aspect of the invention are capable of expressing the coumermycin dimerization domain of E. coli gyrase B (GyrB, residues 1-220) fused to the catalytic domain of mammalian dsRNA dependent protein kinase, PKR (residues 258-551). By flanking this coding region with homologous recombination sites, the construct can be used, for example, for gene insertion by homologous recombination into a locus of interest. When the GyrB-PKR fusion protein is expressed in the presence of coumermycin, the gyrase domain binds to coumermycin in a 2:1 stoichiometric ratio thereby causing the dimerization of the PKR kinase domain (FIG. 1A) (10). This dimerization mimics the endogenous activation of PKR and results in the phosphorylation of translation initiation factor, eIF2α which leads to an inhibition of protein synthesis (11,12). The GyrB-PKR system uses a relatively innocuous antibiotic, coumermycin, and results in the coumermycin dependent shutdown of protein synthesis. This system therefore provides a superior method of isolating clones in a safe and effective manner, without increasing the chances of generating alterations of DNA elsewhere in the genome of the organism.

The first nucleic acid encoding GyrB-PKR can be any nucleic acid sequence capable of expressing the protein of SEQ ID NO: 4 (full length GyrB-PKR), or a functional equivalent thereof. In one embodiment, the first nucleic acid comprises or consists of the nucleotide sequence according to SEQ ID NO: 3 (full length GyrB-PKR NA sequence), or functional equivalents thereof. In another embodiment the first nucleic acid comprises a nucleotide sequence according to SEQ ID NO:1 (encoding the coumermycin binding domain of gyrase) and a nucleic acid sequence capable of expressing the PKR activation domain. In another embodiment the first nucleic acid comprises a nucleotide sequence according to SEQ ID NO:2 (encoding the PKR activation domain) and a nucleic acid sequence capable of expressing the coumermycin binding domain of gyrase.

The first and second site directed homologous recombination sites do not recombine with each other, and flank the first nucleic acid, so that recombination events result in complete elimination of the first nucleic acid from the construct. As used herein, “flanking” means that the first nucleic acid sequence is located completely between the first and second recombination sites, but does not require that the first and second recombination sites are immediately adjacent to the first nucleic acid sequence. A spacer nucleic acid region of any suitable length may be located between the first nucleic acid and either or both the first and second recombination sites. In one embodiment, such spacer regions can be 0-1000 nucleotides; in various further embodiments, 0-500, 0-250, 0-100, 0-50, 0-25, 0-10, 0, 1, 2, 3, 4, 5, 6, 7, 8, 9, or 10 nucleotides in length. As used herein, a “recombination site” is any site that that is identical to a nucleic acid sequence flanking the site of insertion for the gene of interest; the recombination site can be of any length suitable to promote such recombination. Any recombination sites can be used that are suitable for a given intended use, including but not limited to sequences surrounding the vaccinia virus E3L gene, as described in the examples that follow In some embodiments, the recombination site can be a site-directed recombination site, which is a discrete section or segment of DNA that is recognized and bound by a site-specific recombination protein during the initial stages of integration or recombination. Exemplary site-directed recombination sites include, but are not limited to, att sites (including, but not limited to, attB sites, attP sites, attL sites, attR sites, and the like), lox sites (including, but not limited to, loxP sites, loxP511 sites, and the like), psi sites, dif sites, cer sites, frt sites, and mutants, variants, and derivatives of these recombination sites that retain the ability to undergo recombination.

In one embodiment, the nucleic acid construct further comprises a second nucleic acid encoding a second selection marker operatively linked to the nucleic acid encoding GyrB-PKR, wherein the first homologous recombination site and the second homologous recombination site flank the second nucleic acid. The second selection marker can be a positive selection marker (including but not limited to antibiotic resistance genes and enzymatic markers such as lacZ), or a further negative selection marker (ie: a “death gene” encoding a further toxic protein). In one non-limiting example, an antibiotic resistance gene can be selected from either bacterial or eukaryotic genes, and can promote resistance to ampicillin, kanamycin, tetracycline, chloramphenicol, and others known in the art. In another non-limiting example, a second death gene can be any suitable death gene, including but not limited to, rpsL, tetAR, pheS, thyA, lacY, gata-1, ccdB, and sacB. The second death gene can also be selected from either prokaryotic or eukaryotic toxic genes, depending on the selection strategy employed. In these embodiments, the two (or more) selectable markers are preferably present as a “selection cassette” that is flanked by the first and second homologous recombination sites, such that, when used for homologous recombination, a clone of interest inserting via recombination will replace the selection cassette.

In a further embodiment, the first homologous recombination site is located at one end of the construct, and the second homologous recombination site is located at the other end of the construct.

The nucleic acid construct can be RNA or DNA, preferably DNA. The nucleic acid construct may contain other nucleic acid regions of interest, such as control sequences to direct expression of the first nucleic acid and to the second nucleic (if present).

The nucleic acid constructs of the invention are useful, for example, for the construction of expression/recombination vectors for use in cloning and gene targeting. Thus, in a preferred embodiment, the nucleic acid constructs of any embodiment or combination of embodiments above are present in an expression vector, wherein the expression vector comprises nucleic acid control sequences operatively linked to the nucleic acid construct. As used herein, “operatively linked” refers to an arrangement of the nucleic acid sequences wherein they are configured so that they function as a unit for their intended purpose. Any suitable control sequences can be used, so long as they are capable of directing expression of the proteins encoded by the first nucleic acid and, if present, the second nucleic acid. Thus, the control sequences comprise at least one or more transcription or translation sites or signals. In various further embodiments, the vectors may further comprise one or more transcription or translation termination sites, one or more topoisomerase recognition sites, one or more topoisomerases, one or more origins of replication, one or more primer recognition sites, nucleic acid sequences encoding epitope tags, etc.

In accordance with the invention, any vector may be used to construct the vectors of invention. In particular, vectors known in the art and those commercially available (and variants or derivatives thereof) may in accordance with the invention be engineered to include one or more nucleic acid molecules encoding one or more recombination sites (or portions thereof), or mutants, fragments, or derivatives thereof, for use in the methods of the invention. Such vectors may be obtained from, for example, Vector Laboratories Inc.; Promega; Novagen; New England Biolabs; Clontech; Roche; Pharmacia; EpiCenter; OriGenes Technologies Inc.; Stratagene; Perkin Elmer; Pharmingen; and Invitrogen Corp., Carlsbad, Calif. Such vectors may then for example be used for cloning or subcloning nucleic acid molecules of interest. General classes of vectors of particular interest include prokaryotic and/or eukaryotic cloning vectors, Expression Vectors, fusion vectors, two-hybrid or reverse two-hybrid vectors, shuttle vectors for use in different hosts, mutagenesis vectors, transcription vectors, and the like.

In one preferred embodiment, the vectors are plasmid-based. In another preferred embodiment, the vectors are viral-based. Particular vectors of interest include prokaryotic Expression Vectors such as pcDNA II, pSL301, pSE280, pSE380, pSE420, pTrcHisA, B, and C, pRSET A, B, and C (Invitrogen Corp., Carlsbad, Calif.), pGEMEX-1, and pGEMEX-2 (Promega, Inc.), the pET vectors (Novagen, Inc.), pTrc99A, pKK223-3, the pGEX vectors, pEZZ18, pRIT2T, and pMC1871 (Pharmacia, Inc.), pKK233-2 and pKK388-1 (Clontech, Inc.), and pProEx-HT (Invitrogen Corp., Carlsbad, Calif.) and variants and derivatives thereof. Other vectors of particular interest include pUC18, pUC19, pBlueScript, pSPORT, cosmids, phagemids, YACs (yeast artificial chromosomes), BACs (bacterial artificial chromosomes), MACs (mammalian artificial chromosomes), pQE70, pQE60, pQE9 (Quiagen), pBS vectors, PhageScript vectors, BlueScript vectors, pNH8A, pNH16A, pNH18A, pNH46A (Stratagene), pcDNA3 (Invitrogen, Carlsbad, Calif.), pGEX, pTrsfus, pTrc99A, pET-5, pET-9, pKK223-3, pKK233-3, pDR540, pRIT5 (Pharmacia), pSPORT1, pSPORT2, pCMVSPORT2.0 and pSV-SPORT1 (Invitrogen Corp., Carlsbad, Calif.) and variants or derivatives thereof. It will be understood by one of ordinary skill that the present invention also encompasses other vectors not specifically designated herein, which comprise one or more of the isolated nucleic acid molecules used in the invention encoding one or more recombination sites or portions thereof (or mutants, fragments, variants or derivatives thereof), and which may further comprise one or more additional physical or functional nucleotide sequences described herein which may optionally be operably linked to the one or more nucleic acid molecules encoding one or more recombination sites or portions thereof. Such additional vectors may be produced by one of ordinary skill according to the guidance provided in the present specification.

In various preferred embodiments, a suitable viral-based expression vector can be used, including but not limited to viral expression systems derived from vaccinia virus, retroviruses (including but not limited to lentivrus such as HIV, FIV, and SIV), adenovirus, alphavirus, herpes virus, and poxvirus.

The expression vectors of the invention are useful, for example, in carrying out the methods of the invention as described herein. In one non-limiting embodiment, the vector comprises a first nucleic acid encoding GyrB-PKR flanked by the first and second recombination sites. The vector is designed such that a DNA insert of interest will replace the first nucleic acid between the two flanking sites. If the DNA fragment of interest is present in the vector after homologous recombination and culturing of the cells in coumermycin-containing media, the cells containing the vector survive, as the GyrB-PKR encoding nucleic acid will no longer be present on the desired recombinant vector. If the insert of interest is not present, the GyrB-PKR encoding nucleic acid will prevent survival of the cell carrying the undesired vector. Thus, only cells containing positive clones with the DNA fragment of interest will be viable, and easily selected for.

In another non-limiting example, the vector comprises a dual selection cassette comprising the first nucleic acid encoding GyrB-PKR as well as a second selection marker encoding an antibiotic resistance gene under control of at least one promoter, both the first and second nucleic acid being flanked by the first and second recombination sites. After homologous recombination with an insert of interest and culturing of cells in the presence of coumermycin, positive clones for insertion will be antibiotic sensitive and viable (GyrB-PKR negative), due to the replacement of the dual selection cassette with the DNA fragment of interest.

Expression vectors and methods for their engineering and isolation are well known in the art, or they can be obtained through a commercial vendor, such as Invitrogen (Carlsbad, Calif.), Promega (Madison, Wis.), or Statagene (La Jolla, Calif.) and modified as needed. Vector components, control nucleic acids, etc. are typically available from a commercial source or can be isolated from a natural source or prepared using a synthetic means such as PCR. Any arrangement of such components as suitable for a given purpose can be used.

In a further aspect, the present invention provides host cells comprising the expression vectors or nucleic acid constructs of the invention. The host cell can be prokaryotic (such as E. coli) or eukaryotic (algal, fungal, insect, invertebrate, plant, or mammalian host cells) and can be used, for example, in generating large amounts of the expression vectors and nucleic acid constructs, or in carrying out the methods of the invention.

In one embodiment, the host cell contains a nucleic acid construct of the invention stably integrated in the chromosome under the control of a suitable control sequence. In one non-limiting example, this embodiment provides the ability to study functions of individual genes. For example, cellular defense proteins that are upregulated during virus infections can be studied individually, separately from other induced proteins. Though it is commonplace to study gene function through transient expression vectors (plasmids encoding the proteins), most of these plasmids activate host defense pathways and therefore complete function of the encoded protein can be misleading and difficult to understand. In another embodiment, the host cell contains a nucleic acid construct of the invention in an expression vector under the control of a suitable control sequence.

In a further aspect, the present invention provides kits comprising:
(a) one or more nucleic acid constructs, expression vectors, and/or host cells according to any embodiment or combination of embodiments of the invention; and
(b) a cloning vector, comprising the first homologous recombination site and the second homologous recombination site flanking a cloning site.

The cloning vector can be any suitable vector, including but not limited to the vector constructs disclosed above. The cloning vector comprises recombination sites that can be used in combination with the recombination sites on the nucleic acid constructs and expression vectors of the invention to promote homologous recombination, and thus transfer an insert of interest cloned into the cloning site into the nucleic acid construct or expression vector of the invention. The cloning site comprises one or more regions suitable for cloning an insert of interest into the cloning site, including but not limited to one or more restriction sites unique to the cloning vector. Any nucleic acid insert of interest can be cloned into the cloning site for subsequence homologous recombination.

In a further aspect, the present invention provides methods for homologous recombination, comprising (a) expressing in a host cell a first expression vector or nucleic acid construct according to any embodiment or combination of embodiments of the invention; (b) expressing in the host cell a second expression vector comprising a gene of interest flanked by the first homologous recombination site and the second homologous recombination site; and (c) culturing the cells in medium comprising coumermycin under conditions suitable to cause host cell death if homologous recombination has not occurred.

The methods of the invention comprises use of GyrB-PKR, containing the antibiotic coumermycin binding domain of Escherichia coli gyrase subunit B fused to the activation domain of human protein kinase R, PKR, as a general negative selectable marker for the generation of genetic recombinants. In the presence of the antibiotic coumermycin, the protein is activated and leads to cell death. Cells expressing this gene are susceptible to the negative effects of coumermycin. If GyrB-PKR is replaced by a recombinant gene of interest, the clones will be resistant to the effects of coumermycin, therefore resulting in the enrichment of recombinants by the loss of the coumermycin resistance (cmr) gene. This negative selection system acts on the level of protein synthesis, universal to all eukaryotic organisms, and avoids the use of special cell lines or noxious mutagenic chemicals that can be potentially damaging to genomic DNA. Furthermore, no extraneous marker sequence is left in the genetic locus of interest.

In one embodiment, the host cell can be one that has been transfected or infected/transduced with an expression vector according to any embodiment or combination of embodiments of the invention. In another embodiment, the host cell may comprise a nucleic acid construct of any embodiment or combination of embodiments of the invention stably integrated in the chromosome under the control of a suitable control sequence, where the control sequence may be a naturally occurring host cell chromosome, or may be introduced as part of the nucleic acid construct.

Any suitable gene of interest can be used with the second expression vector, so long as it is flanked by the first homologous recombination site and the second homologous recombination site. Any suitable recombination sites can be used, as discussed above.

Any suitable growth conditions can be used that lead to expression of GyrB-PKR from the first expression vector, and that include suitable amounts of coumermycin in the media to activate GyrB-PKR. In on embodiment, a coumermycin concentration range of about 5 ng/ml to about 500 ng/ml can be used. It is well within the level of those of skill in the art to choose such suitable conditions based on the teachings herein. The methods are conducted under conditions suitable to promote homologous recombination, and wherein the first expression vector comprises a recombinant viral vector, wherein the contacting occurs under conditions suitable to promote infection of the host cell with the recombinant virus and under conditions suitable to promote homologous recombination between the second expression vector nucleic acid and the recombinant virus nucleic acid.

In this embodiment, any recombinant virus can be used, including but not limited to recombinant vaccinia virus, retroviruses (including but not limited to lentivrus such as HIV, FIV, and SIV), adenovirus, alphavirus, herpes virus, and poxvirus.

The methods of the invention can be used with any system, both DNA and RNA, that undergoes homologous recombination. This would include, but not be limited to, the isolation of recombinant viruses to the more complex recombinant ES cell clones. Current methods utilizing negative selection include positive/negative selection (PNS), to screen against random non-homologous recombination, and the double replacement and ‘hit and run’ strategies, both of which are variations of two-step replacement methods to introduce subtle mutations in ES cells and foreign genes in non-mammalian genomes. In all cases, the GyrB-PKR method is an improvement over the current methods. Clearly, a method to isolate homologous recombinants without the requirement of mutagenic agents, special cell lines or the retention of a marker gene is necessary. The GyrB-PKR system has proven to be an effective means to remove selection marker in a rapid manner, as demonstrated in the vaccinia virus (VACV) system shown below. Furthermore, this method is novel in that it inhibits protein synthesis, decreasing the probability of generating random mutations in replicating genome and is applicable to most organisms using the eukaryotic translation machinery.

The methods of the invention can be used, for example, in vaccine development. For example, the methods can be used for high throughput antigen screening and vaccine development, as the methods provide the ability to rapidly test the proteome of any pathogen for protective antigens to be used in vaccine development.

In another embodiment, the methods of the invention can be used for homologous recombination in a genome, for example, to knock-out the function of a gene in a cell, or to confer a novel phenotype on the cell. The method can further be used to produce a transgenic non-human organism (including but not limited to mouse, rat, primate, etc.) having the recombined nucleic acid insert stably maintained in its genome, in embodiments using the host cell comprising a nucleic acid construct of any embodiment or combination of embodiments of the invention stably integrated in the chromosome under the control of a suitable control sequence.

### EXAMPLES

### Abstract

Vaccinia virus has been a powerful tool in molecular biology and vaccine development. The relative ease of inserting and expressing foreign genes combined with its broad host range has made vaccinia virus an attractive antigen delivery system against many heterologous diseases. Many different approaches have been developed to isolate recombinant vaccinia virus generated from homologous recombination, however, most are time consuming, often requiring a series of passages or specific cell lines. Here, we introduce a rapid method for isolating recombinants using the antibiotic coumermycin and the interferon-associated PKR pathway to select for vaccinia virus recombinants. This method uses a negative selection marker in the form of a fusion protein, GyrB-PKR, consisting of the coumermycin dimerization domain of Escherichia coli gyrase subunit B fused to the catalytic domain of human PKR. Coumermycin dependent dimerization of this protein results in activation of PKR and the phosphorylation of translation initiation factor, eIF2α. Phosphorylation of this factor leads to an inhibition of protein synthesis, and an inhibition of virus replication. In the presence of coumermycin, recombinants are isolated due to the loss of this coumermycin sensitive gene by homologous recombination. We demonstrate that this method of selection is highly efficient and requires limited rounds of enrichment to isolate recombinant virus.

### Introduction

Vaccinia virus (VACV), the poxvirus used as the vaccine against smallpox, has gained widespread use as a general vector for expressing foreign proteins in mammalian cells. The ability to take up large inserts of DNA and express high levels of foreign protein in a wide variety of cell lines has made VACV an attractive delivery vehicle for expressing antigens and analyzing protein function (1). The standard method of generating VACV recombinants is through homologous recombination between replicating viral DNA and a transfected plasmid or PCR product containing the protein coding sequence of interest “flanked” by viral sequences (1). The frequency of recombination accounts for approximately 0.1% of total virus and isolation of the recombinants typically requires a series of selections using a genetic marker (1,2).

Many different selection schemes have been developed for detection of VACV recombinants. Such examples include the use of thymidine kinase negative and positive selection (3,4), reversal of host range mutations (5-7), neomycin resistance (8) and transient dominant selections using mycophenolic acid (MPA)(1). These methods have proven to be quite efficient, however they often require special cell lines or serial passages in selection media and therefore can be time consuming.

Here we demonstrate a novel, alternative method for rapid isolation of recombinant VACV using the antibiotic coumermycin and a coumermycin sensitive VACV, VACVΔE3L::GyrB-PKR. The VACVΔE3L::GyrB-PKR expresses the coumermycin dimerization domain of E. coli gyrase B (GyrB, residues 1-220) fused to the catalytic domain of mammalian dsRNA dependent protein kinase, PKR (residues 258-551), in the viral locus of interest for gene insertion. The GyrB-PKR system has been characterized previously (9). In this heterologous system, the gyrase domain binds to coumermycin in a 2:1 stoichiometric ratio thereby causing the dimerization of the PKR kinase domain (FIG. 1A) (10). This dimerization mimics the endogenous activation of PKR and results in the phosphorylation of translation initiation factor, eIF2α which leads to an inhibition of protein synthesis (11,12). For this study, we used the E3L locus of VACV to express GyrB-PKR. In this system, recombinants are generated by standard homologous recombination between a plasmid containing the insert of interest surrounded by sequences that flank the E3L gene and the parental VACVΔE3L::GyrB-PKR. Recombinants are resistant to the effects of coumermycin, whereas the replication of VACVΔE3L::GyrB-PKR is inhibited following treatment with coumermycin. Therefore, this system exclusively allows the viability of viruses that undergo a double recombination event that result in loss of the GyrB-PKR gene and retention of the desired insert.

### Materials and Methods

### Cells, Virus and Reagents

Baby hamster kidney (BHK-21-CCL10) and rabbit kidney cells (RK13-CCL37) (ATCC, Manassas, Va., USA) used in the experiments were maintained in MEM (Mediatech, Manassas, Va., USA) supplemented with 5% FBS (HyClone Logan, Utah, USA) and 10 μg/mL gentamicin (Invitrogen, Carlsbad, Calif., USA) at 37° C. with 5% CO₂. Coumermycin A1 (Sigma, St. Louis, Mo., USA) was dissolved in DMSO (Invitrogen) at a stock concentration of 5 mg/ml and diluted with phosphate buffered saline (PBS). Vectors pC939 and pC940 containing GyrB-PKR and GyrB-PKRK296H, respectively, were kindly provided by Tom Dever and has been described (9). These vectors contain the residues 1-220 of E. coli Gyrase B fused to the kinase domain (residues 258-551) of human PKR. The catalytic inactive mutant contains a lysine to histidine mutation at residue 296 of the PKR domain. Viruses used in this study expressed the GyrB-PKR or its mutant protein in either the E3L or the J2R locus. Isolation of VACV expressing GyrB-PKR was by transient dominant selection using mycophenolic acid as describe previously (13,14). Individual mycophenolic acid resistant plaques were tested for sensitivity to coumermycin, and the most coumermycin sensitive viruses were further characterized. VACV strains used were Western Reserve (WR) and NYVAC. All infections, plaque purifications, virus amplifications and viral genomic extraction for sequencing were carried out as previously described (14).
Coumermycin sensitivity of VACVΔE3L, VACVΔE3L::GyrB-PKR, VACVΔE3L::GyrB-PKRK296H, and VACVΔJ2R::GyrB-PKR RK13 cells grown in 6-well dishes were treated with coumermycin A1 at doses ranging from 0-100 ng/mL in media for 16 hours. The cells were infected with 100 pfu of VACVΔE3L, VACVΔE3L::GyrB-PKR, VACVΔE3L::GyrB-PKRK296H and the infections were carried out in media supplemented with the corresponding concentration of coumermycin for 48 hours. To test the sensitivity of the VACV4J2R::GyrB-PKR to coumermycin, RK13 cells were mock treated or treated with coumermycin A1 at a concentration of 100 ng/mL for 24 hours and then infected with tenfold serial dilutions of the virus stock. Infections were carried out for 48 hours in media with or without 100 ng/mL of coumermycin A1. Cells were stained with crystal violet and plaques were analyzed by a standard plaque assay.
Western Blot Analysis for GyrB-PKR and eIF2a Phosphorylation
For GyrB-PKR expression analysis, subconfluent RK13 cells were mock treated or treated with 100 ng/mL coumermycin A1. After 16 hours, the cells were infected with VACVΔE3L, VACVΔE3L::GyrB-PKR or VACVΔE3L::GyrB-PKRK296H at an MOI of 5 pfu/cell. At 6 hours post infection, the cells were scraped into media and pelleted by centrifugation at 500×g for 10 minutes at 4° C. The cells were lysed by addition of RIPA lysis buffer (1×PBS, 0.1% sodium dodecyl sulfate, 1% NP-40, 0.5% sodium deoxycholate, 100 mM sodium fluoride) followed by incubation on ice for 10 minutes. The lysates were centrifuged at 10,000×g for 10 minutes at 4° C. and the supernatant transferred to new tube. Equal amounts of 2×SDS loading buffer was added to the lysates and then separated on a 12% SDS PAGE gel under denaturing conditions and then transferred onto PVDF membrane. Following transfer, the membranes incubated in blocking buffer (3% nonfat dry milk, 140 mM NaCl, 3 mM KCl, 20 mM Tris pH 7.8, 0.05% Tween 20) for 1 hour at room temperature. GyrB-PKR expression was determined by using rabbit antibodies directed against total PKR and the phosphorylation of eIF2α by phospho specific eIF2α antibodies (Cell Signaling, Danvers, Mass., USA) at a concentration of 1:1000 diluted in blocking buffer. Secondary goat anti-rabbit antibodies conjugated to horseradish peroxidase (Sigma) were added at 1:15,000 in blocking buffer followed by chemiluminescent development (Pierce Supersignal Dura).

### Generation of VACVΔE3L::LacZ by Coumermycin System

In vivo recombination (IVR) of the following viruses occurred accordingly: Subconfluent BHK cells (2×10⁶ cells total) were transfected with 2 ug of pMPLacZ using Lipofectamine and Plus Reagent (Invitrogen) per the manufacturer's directions and coinfected with parental VACVΔE3L::GyrB-PKR at an MOI of 0.05 pfu/cell. At 30 hours post infection, cells were scraped and virus was released by three rounds of freeze-thaw treatment followed by sonication. RK13 cells pretreated with or without coumermycin A1 at 100 ng/ml for 16 hours were infected with dilutions of the IVR extract, and overlaid with media containing coumermycin if required. After 48 hours post infection, the cells were covered with an agarose overlay consisting of 1×MEM, 1.5% agarose, X-gal substrate (10 mg/mL) and neutral red. At 24 hours post infection, recombinant plaques (blue) were quantified and compared to the number of parental plaques (clear).

### Results and Discussion

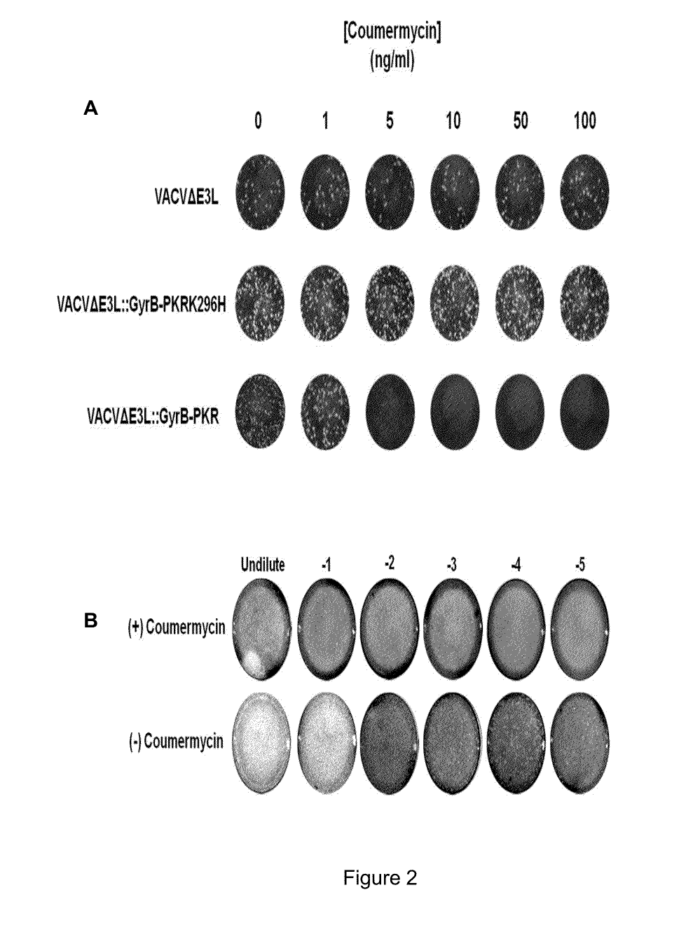

The parental virus (VACVΔE3L::GyrB-PKR) was constructed to express the first 220 amino acids of E. coli GyrB fused to residues 258-551 of the kinase domain of human PKR in the E3L locus of VACV. As described in Materials and Methods, GyrB-PKR was inserted into VACVΔE3L by transient dominant selection for mycophenolic acid resistance. Since the kinase activity of this PKR fusion protein is dependent on interaction with coumermycin (9), this virus would be expected to be coumermycin sensitive (cmr^{s}). In addition to the coumermycin sensitive virus, a PKR catalytically inactive mutant, VACVΔE3L::GyrB-PKRK296H, was constructed which was unable to phosphorylate eIF2α. To confirm that the viruses were able to express the chimeric proteins, RK13 cells were infected with VACVΔE3L::GyrB-PKR, VACVΔE3L::GyrB-PKR K296H, and VACVΔE3L and extracts prepared at 6 hours post infection. Immunoblot analysis using antibodies against PKR illustrate that only VACVΔE3L::GyrB-PKR and VACVΔE3L::GyrB-PKRK296H viruses express the GyrB-PKR protein (FIG. 1B). To determine whether the GyrB-PKR system was able to function in the context of a vaccinia virus infection in cells in culture, the sensitivity of VACVΔE3L::GyrB-PKR, VACVΔE3L and VACVΔE3L::GyrB-PKRK296H to coumermycin was compared. RK13 cells were pretreated with increasing concentrations of coumermycin and infected with 50-100 pfu of each virus (FIG. 2A). For VACVΔE3L::GyrB-PKR, sensitivity to coumermycin was observed at concentrations greater than 5 ng/ml. As expected, both VACVΔE3L and the catalytically inactive mutant, VACVΔE3L::GyrB-PKRK296H, were resistant to all doses of coumermycin thereby supporting that the observed sensitivity of VACVΔE3L::GyrB-PKR to coumermycin was dependent on the function of the chimeric protein, GyrB-PKR. To determine if the coumermycin sensitive phenotype was not limited to the expression of the protein in the E3L locus, we also tested the sensitivity to coumermycin with VACV expressing the GyrB-PKR protein in the J2R (thymidine kinase) locus. Clearly this recombinant virus was sensitive to coumermycin by at least 100,000 fold when compared to untreated cells (FIG. 2B), demonstrating the versatility of this system for selection of recombinant viruses.

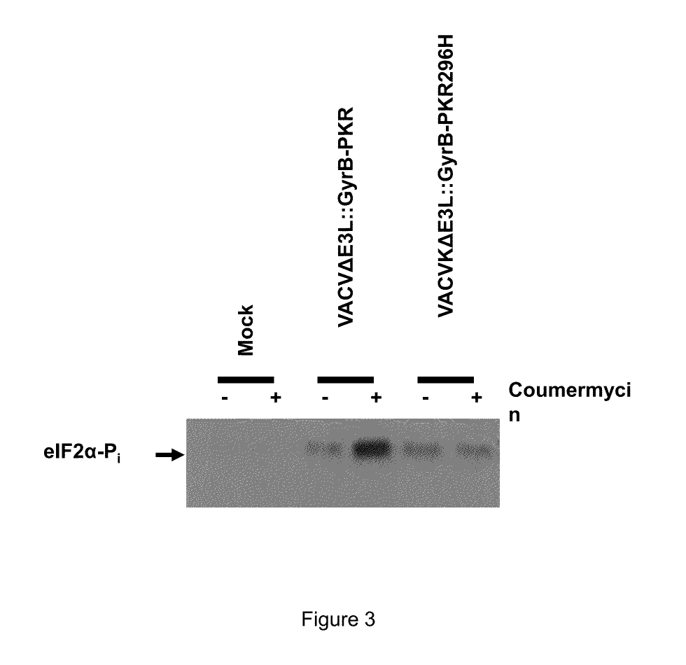

It has been established that the treatment of coumermycin results in the dimerization and activation of GyrB-PKR and leads to the subsequent inhibition of translation (9,15). This inhibition occurs by phosphorylation of a key mediator of translation, eIF2α. To determine that the sensitivity of VACVΔE3L::GyrB-PKR to coumermycin was the result of eIF2α phosphorylation, the level of eIF2α phosphorylation between the virus infections in the presence or absence of coumermycin was determined. RK13 cells treated with or without coumermycin at 100 ng/ml for 16 hours were infected at an MOI of 5 to ensure all cells were infected. At 6 hours post infection, extracts were prepared and assayed for eIF2α phosphorylation. FIG. 3 shows that without coumermycin, infection with either VACVΔE3L::GyrB-PKR or VACVΔE3L::GyrB-PKRK296H led to low levels of eIF2α phosphorylation compared to mock infected cells. The low levels of phosphorylation could be attributed to the loss of the E3L gene, which acts to sequester viral dsRNA preventing endogenous PKR activation during a VACV infection (13, 16). Comparatively, in the presence of coumermycin, infection with VACVΔE3L::GyrB-PKR infection led to an increase in eIF2α phosphorylation. As expected, infection with the catalytically inactive mutant, VACVΔE3L::GyrB-PKRK296H, did not lead to an increase in eIF2α phosphorylation following coumermycin treatment. These results suggest that coumermycin mediated dimerization and activation of GyrB-PKR effectively increased eIF2α phosphorylation and therefore resulted in the sensitivity of VACVΔE3L::GyrB-PKR to the antibiotic.

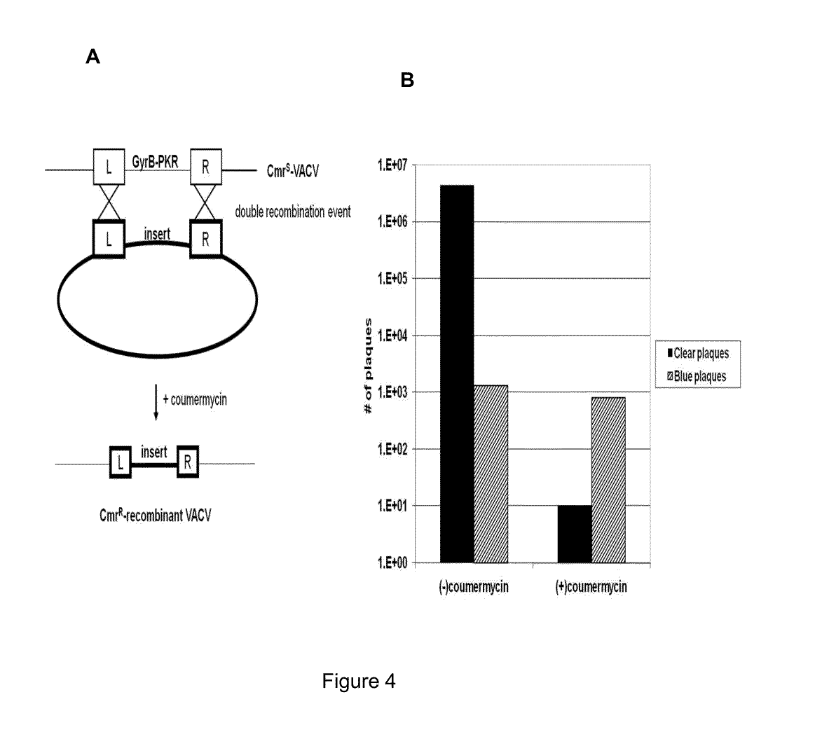

Next, the VACVΔE3L::GyrB-PKR system was tested for the ability to isolate recombinant viruses. Ideally, the antibiotic should effectively inhibit the replication of viruses that do not contain the desired insert and allow only recombinant viruses to replicate (FIG. 4A). pMPAE3L-lacZ was used as the donor plasmid, which upon recombination would place the β-galactosidase gene in the E3L locus, in place of GyrB-PKR. This would allow for screening and quantitation of the recombinants in the presence of X-gal substrate, resulting in blue plaques. In the absence of coumermycin, the ratio of parental virus to recombinants was approximately 4000:1 (4×10⁶ parental to 1×10³ recombinants), yielding a recombination efficiency of 0.025% (FIG. 4B). Upon treatment with coumermycin, only 10 parental viruses were detected. However, the number of recombinants detected with or without coumermycin treatment was comparable, giving 1000 recombinant plaques without coumermycin to 800 plaques with coumermycin treatment. In the presence of coumermycin, the ratio of parental virus to recombinant virus was approximately 1:80 (10 parental to 800 recombinants). It should be noted that the plaque morphology of the parental virus differed from the recombinant VACVΔE3L::LacZ in the presence of coumermycin where VACVΔE3L::GyrB-PKR plaques appeared as distinct foci in the monolayer, rather than forming clear plaques free of cells. This phenotype only occurred in the presence of the antibiotic. VACVΔE3L::LacZ, a well-characterized virus with a very limited host range, formed plaques easily distinguishable from VACVΔE3L::GyrB-PKR in the presence of coumermycin (data not shown). Individual plaques picked after coumermycin selection routinely yielded only blue recombinant viruses upon re-plaquing (zero non-blue plaques out of 213 second round plaques assayed). This demonstrates that this selection method can be used to obtain pure virus cultures after minimal rounds of plaque purification. Overall, these results demonstrate that this system is highly efficient in generating recombinant viruses containing genes of interest in a rapid manner. It should be noted that while in this example a plasmid containing the gene of interest flanked by homologous arms was used as the donor for homologous recombination. Since the donor DNA does not need to contain any extraneous sequences, a PCR product containing the gene of interest flanked by homologous arms can be used as the DNA donor using this method.

This study demonstrated that the coumermycin/GyrB-PKR negative selection method is an effective system for the isolation of recombinant VACV viruses. Once a coumermycin sensitive parental virus was obtained, pure recombinant viruses were rapidly isolated following minimal rounds of plaque purification. The sensitivity to coumermycin was not limited to the GyrB-PKR gene being expressed from the E3L locus as this phenotype was maintained when GyrB-PKR was expressed from the TK locus (FIG. 4), demonstrating the versatility of the system. The GyrB-PKR selection scheme has many advantages over selection methods that result in the rapid isolation of recombinants. Markers such as neomycin (8), hygromycin (17), and puromycin (18), which confer antibiotic resistance, function to enrich the population of recombinants that have taken up the desired DNA through homologous recombination. Enrichment of these recombinants requires multiple rounds of passage and these methods often require the retention of the markers. The use of transient dominant selection markers, such as MPA (1) and GFP-bsd (19), requires multiple passages for selection of marker gene uptake followed by resolution for the desired recombinant. In addition, the GyrB-PKR system does not require special cells for the isolation of recombinants unlike thymidine kinase selection (3, 4). Furthermore, coumermycin is relatively innocuous to eukaryotic cells, unlike other systems such as MPA and herpes simplex thymidine kinase, which use nucleoside analogs that are potentially mutagenic.

### REFERENCES

1. Falkner, F. G. and B. Moss. 1990. Transient dominant selection of recombinant vaccinia viruses. Journal of Virology 64:3108-3111.
2. Ball, L. A. 1987. High-frequency homologous recombination in vaccinia virus DNA. Journal of Virology 61:1788-1795.
3. Mackett, M., G. L. Smith, and B. Moss. 1984. General method for production and selection of infectious vaccinia virus recombinants expressing foreign genes. Journal of Virology 49:857-864.
4. Weir, J. P., G. Bajszar, and B. Moss. 1982. Mapping of the vaccinia virus thymidine kinase gene by marker rescue and by cell-free translation of selected mRNA. Proceedings of the National Academy of Sciences of the United States of America 79:1210-1214.
5. Holzer, G. W., W. Gritschenberger, J. A. Mayrhofer, V. Wieser, F. Dorner, and F. G. Falkner. 1998. Dominant host range selection of vaccinia recombinants by rescue of an essential gene. Virology 249:160-166.
6. Perkus, M. E., K. Limbach, and E. Paoletti. 1989. Cloning and expression of foreign genes in vaccinia virus, using a host range selection system. Journal of Virology 63:3829-3836.
7. Staib, C., I. Drexler, M. Ohlmann, S. Wintersperger, V. Erfle, and G. Sutter. 2000. Transient host range selection for genetic engineering of modified vaccinia virus Ankara. BioTechniques 28:1137-1142, 1144-1136, 1148.
8. Franke, C. A., C. M. Rice, J. H. Strauss, and D. E. Hruby. 1985. Neomycin resistance as a dominant selectable marker for selection and isolation of vaccinia virus recombinants. Molecular and Cellular Biology 5:1918-1924.
9. Ung, T. L., C. Cao, J. Lu, K. Ozato, and T. E. Dever. 2001. Heterologous dimerization domains functionally substitute for the double-stranded RNA binding domains of the kinase PKR. The EMBO journal 20:3728-3737.
10. Ali, J. A., A. P. Jackson, A. J. Howells, and A. Maxwell. 1993. The 43-kilodalton N-terminal fragment of the DNA gyrase B protein hydrolyzes ATP and binds coumarin drugs. Biochemistry 32:2717-2724.

11. Clemens, M. J. 1997. PKR—a protein kinase regulated by double-stranded RNA. Int J Biochem Cell Biol 29:945-949.
12. Hershey, J. W. 1991. Translational control in mammalian cells Annual Review of Biochemistry 60:717-755.
13. Jacobs, B. L., J. O. Langland, and T. Brandt. 1998. Characterization of viral double-stranded RNA-binding proteins. Methods San Diego, Calif. 15:225-232.
14. Kibler, K. V., T. Shors, K. B. Perkins, C. C. Zeman, M. P. Banaszak, J. Biesterfeldt, J. O. Langland, and B. L. Jacobs. 1997. Double-stranded RNA is a trigger for apoptosis in vaccinia virus-infected cells. Journal of Virology 71:1992-2003.
15. Friedrich, I., M. Eizenbach, J. Sajman, H. Ben-Bassat, and A. Levitzki. 2005. A cellular screening assay to test the ability of PKR to induce cell death in mammalian cells. Mol Ther 12:969-975.
16. Langland, J. O. and B. L. Jacobs. 2004. Inhibition of PKR by vaccinia virus: role of the N- and C-terminal domains of E3L. Virology 324:419-429.
17. Zhou, J., L. Crawford, X. Y. Sun, and I. H. Frazer. 1991. The hygromycin-resistance-encoding gene as a selection marker for vaccinia virus recombinants. Gene 107:307-312.
18. Sanchez-Puig, J. M. and R. Blasco. 2000. Puromycin resistance (pac) gene as a selectable marker in vaccinia virus. Gene 257:57-65.
19. Wong, Y. C., L. C. Lin, C. R. Melo-Silva, S. A. Smith, and D. C. Tscharke. Engineering recombinant poxviruses using a compact GFP-blasticidin resistance fusion gene for selection. J Virol Methods 171:295-298.

We claim:

1. A method for homologous recombination, comprising
(a) expressing in a host cell a first nucleic acid encoding a coumermycin dimerization domain of E. coli gyrase B fused to a catalytic domain of mammalian dsRNA dependent protein kinase, PKR (GyrB-PKR), wherein the first nucleic acid is operatively linked to nucleic acid control sequences, and wherein the first nucleic acid is present on a first nucleic acid construct that further comprises:
(i) a first homologous recombination site flanking the first nucleic acid; and
(ii) a second homologous recombination site flanking the first nucleic acid; wherein the first homologous recombination site and the second homologous recombination site are not capable of recombining with each other;
(b) expressing in the host cell a second expression vector comprising a gene of interest flanked by the first homologous recombination site and the second homologous recombination site, wherein the gene of interest is operatively linked to nucleic acid control sequences; and
(c) culturing the cells in medium comprising coumermycin under conditions suitable to cause host cell death if homologous recombination between the first expression vector and the second expression vector has not occurred.

2. The method of claim 1, wherein the first nucleic acid construct comprises a first expression vector.

3. The method of claim 1, wherein the first nucleic acid construct is stably integrated into the host cell genome.

4. The method of claim 1, wherein the first nucleic acid encodes the GyrB-PKR of SEQ ID NO:4.

5. The method of claim 1, wherein the first nucleic acid construct further comprises a second nucleic acid encoding a second selection marker, wherein the first homologous recombination site and the second homologous recombination site flank the second nucleic acid.

6. The method of claim 3, wherein the second selection marker is a positive selection marker.

7. The method of claim 3, wherein the second selection marker is a negative selection marker.

8. The method of claim 1, wherein the first nucleic acid construct is a DNA construct and the second expression vector is a DNA vector.

9. The method of claim 1, wherein the first nucleic acid construct comprises a first expression vector.

10. The method of claim 9, wherein the first expression vector and the second expression vector are plasmid expression vectors.

11. The method of claim 9, wherein the first expression vector is a viral expression vector.
